(19) United States
(12) Patent Application Publication
(10) Pub. No.: US 2025/0099755 A1
(43) Pub. Date: Mar. 27, 2025

(54) ELECTRICAL STIMULATION DEVICE AND METHOD

(71) Applicant: GIMER MEDICAL CO., LTD., New Taipei City (TW)

(72) Inventors: Wei-Tso Lin, New Taipei City (TW); Kuo-Hsiang Lu, New Taipei City (TW)

(21) Appl. No.: 18/886,476

(22) Filed: Sep. 16, 2024

(30) Foreign Application Priority Data
Sep. 25, 2023 (TW) 112136944
Jun. 4, 2024 (TW) 113120706

(51) Int. Cl. A61N 1/36; A61N 1/04; A61N 1/05

(52) U.S. Cl. CPC A61N 1/36021; A61N 1/0456; A61N 1/0504; A61N 1/36082

(57) ABSTRACT

An electrical stimulation device includes an electrode assembly and an electrical stimulator. The electrical stimulator generates an electrical stimulation signal and is coupled to the electrode assembly. The electrical stimulation signal is transmitted to a target region through the electrode assembly. The electrical stimulation signal contains a plurality of burst signals, and the burst signals have the burst frequency between 0.1 Hz and 1,000 Hz. Each burst signal contains a plurality of pulses, and the pulses have the pulse frequency between 20 kHz (kilohertz) and 1,000 kHz. The electrical stimulation signal does not induce activation of an upstream neural structure or a downstream neural structure of the target region.

### CROSS-REFERENCES TO RELATED APPLICATIONS

This non-provisional application claims priority under 35 U.S.C. § 119 (a) to patent application No. 112136944 filed in Taiwan, R.O.C. on Sep. 25, 2023 and patent application No. 113120706 filed in Taiwan, R.O.C. on Jun. 4, 2024, the entire contents of which are hereby incorporated by reference.

### BACKGROUND

### Technical Field

The present invention relates to the technical field of electrical stimulation, particularly to an electrical stimulation device and method.

### Related Art

Electrical stimulation techniques have a long history of treating or relieving various pains. However, most of the conventional electrical stimulation techniques activate nerve signals through action potentials generated by low-frequency electrical stimulation (40 Hz to 100 Hz) to treat or relieve various pains. The aforementioned conventional electrical stimulation techniques are used to treat or relieve various pains through the gate control theory of pain. The gate control theory of pain is that the electrical stimulation signal activates the nerves to trigger the action potentials, and then the corresponding sensing signals can be measured in the brain to block or interfere the pain signals from transmitting into the central nervous system. When the brain receives the reduced pain signals, the pain sensation is also decreased. In other words, the activation/excitation of nerves conveying pain signals to generate sensory signals via electrical stimulation may interfere with the conduction of the pain signals, thereby treating or relieving pain.

### SUMMARY

However, at present, there is no electrical stimulation technique in which an electrical stimulation signal can regulate nerves without activating the nerves to generate sensory signals. For this purpose, the present disclosure provides an electrical stimulation device and method, which does not induce activation of an upstream neural structure or a downstream neural structure of a target region.

In some embodiments, the electrical stimulation device includes an electrode assembly and an electrical stimulator. The electrical stimulator generates an electrical stimulation signal and is coupled to the electrode assembly. The electrical stimulation signal is transmitted to the target region through the electrode assembly. The electrical stimulation signal contains a plurality of burst signals, and the burst signals have the burst frequency between 0.1 Hz and 1,000 Hz. Each the burst signal contains a plurality of pulses, and the pulses have the pulse frequency between 20 kHz and 1,000 kHz. The electrical stimulation signal does not induce the activation of the upstream neural structure or the downstream neural structure of the target region.

In some embodiments, the electrical stimulation method includes coupling an electrode assembly to the target region, generating an electrical stimulation signal and transmitting the electrical stimulation signal to the electrode assembly to electrically stimulate the target region. The electrical stimulation signal does not induce the activation of the upstream neural structure or the downstream neural structure of the target region. The electrical stimulation signal contains a plurality of burst signals, and the burst signals have the burst frequency between 0.1 Hz and 1,000 Hz. Each the burst signal contains a plurality of pulses, and the pulses have the pulse frequency between 20 kHz and 1,000 kHz.

In some embodiments, the activation of the upstream neural structure is generating an action potential from the upstream neural structure and the activation of the downstream neural structure is generating an action potential from the downstream neural structure.

In some embodiments, the upstream neural structure of the target region is a cranial nerve or spinal cord.

In some embodiments, the downstream neural structure of the target region is a sciatic nerve, a sacral nerve or a peripheral nerve.

In some embodiments, the electrical stimulation signal has a voltage threshold sufficient to desensitizes nerves.

In some embodiments, when the electrical stimulation signal has a voltage intensity greater than 1.5 times of the voltage threshold, the electrical stimulation signal still does not induce the activation of the upstream neural structure or the downstream neural structure of the target region.

In some embodiments, the electrical stimulation signal has the pulse frequency range between 100 kHz and 750 kHz.

In some embodiments, the upstream neural structure or the downstream neural structure is outside 3 cm radius of the target region.

In some embodiments, a cumulative time of the electrical stimulation signal is less than 12 hours per day.

In some embodiments, the electrical stimulation signal is used to inhibit and/or relieve pain.

In summary, the electrical stimulation device or method does not induce the activation of the upstream neural structure or the downstream neural structure of the target region. Under the voltage intensity with a therapeutic effect, the electrical stimulation device or method does not cause the activation of an orthodromic or antidromic neural structure, does not influence a spinal cord loop, does not cause spinal cord injury, does not cause electromyogram signal conduction, and does not cause neurological function defect. The electrical stimulation signal of the electrical stimulation device or method is a “paresthesia-free” electrical stimulation signal, a “paresthesia-independent” electrical stimulation signal, a “subthreshold stimulation” electrical stimulation signal or a “sub-perception” electrical stimulation signal.

### BRIEF DESCRIPTION OF THE DRAWINGS

FIG. 1 is a functional block diagram of the electrical stimulation device according to some embodiments;

FIG. 2A is a schematic diagram of an example of the electrical stimulation device of FIG. 1;

FIG. 2B is a use schematic diagram of an example of the electrical stimulation device of FIG. 1;

FIG. 3 is a schematic diagram of one other example of the electrical stimulation device of FIG. 1;

FIG. 4 is a schematic diagram of another example of the electrical stimulation device of FIG. 1;

FIG. 5A is a schematic diagram of an example of the electrical stimulation signal generated by the electrical stimulator of FIG. 1;

FIG. 5B is a schematic diagram of one other example of the electrical stimulation signal generated by the electrical stimulator of FIG. 1;

FIG. 5C is a schematic diagram of another example of the electrical stimulation signal generated by the electrical stimulator of FIG. 1;

FIG. 6 shows the test results of rat hind paw withdrawal thresholds of an experimental group A;

FIG. 7 shows the test results of rat hind paw withdrawal thresholds of an experimental group B;

FIG. 8 shows the test results of rat hind paw withdrawal thresholds of an experimental group C;

FIG. 9 shows the test results of rat cranial nerve stimulation thresholds;

FIG. 10 shows the test results of rat sciatic nerve stimulation thresholds;

FIG. 11 shows the test results of rat cranial nerve stimulation thresholds; and

FIG. 12 shows the test results of rat sciatic nerve stimulation thresholds.

### DETAILED DESCRIPTION

The term “action potential” herein refers to a change in membrane potential that occurs when a cell membrane is subjected to a rapid, transient and conductive stimulus. When the membrane potential exceeds a neural threshold potential, muscle cells or neurons are caused to generate an action potential. The action potential is a physiological basis for realizing nerve signal conduction, the transmission of the action potential can cause the response of related nervous systems. If the electrical stimulation intensity is lower than the neural threshold potential, the action potential cannot be generated.

The term “neural structure” herein refers to a structure that includes both neural tissue and non-neural tissue. The neural tissue includes neurons. The non-neural tissue includes neurogliocytes, myelin sheaths, immune cells, connective tissues, epithelial cells, cardiovascular cells and/or blood cells and the like. The neurogliocytes include astrocytes, oligodendrocytes, ependymal cells, radial glial cells, Schwann cells, satellite cells, microglial cells and/or pituitary cells and the like.

The term “electrical stimulation signal” herein refers to an electrical signal transmitted by an electrical stimulator through an electrode assembly to an organism. For example, the electrical stimulation signal may be measured by methods of voltage, current, power, etc.

The term “organism” herein may refer to a human or an animal.

The term “target region” herein refers to a neural or non-neural structure that receives the electrical stimulation signal.

The term “nerve or neural structure activation” or “nerve or neural structure activated” herein refers to at least one action potential generated or initiated by and propagated along the nerve or neural structure in response to a stimulus.

It should be understood that, unless explicitly defined otherwise, the terms “a” and “an” herein are not intended to limit only one, and may, under a reasonable range, refer to a single one, any one of a plurality or one kind.

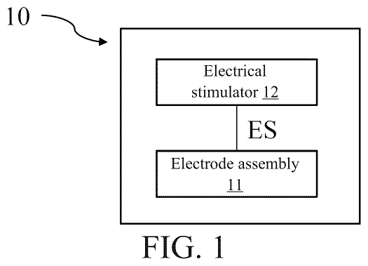

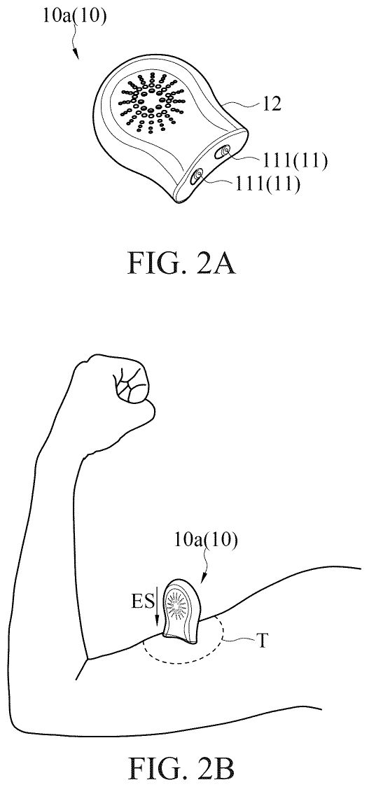

FIG. 1 is a functional block diagram of the electrical stimulation device according to some embodiments. Refer to FIG. 1. An electrical stimulation device 10 includes an electrode assembly 11 and an electrical stimulator 12. The electrical stimulator 12 is coupled to the electrode assembly 11 and generates an electrical stimulation signal ES. The electrode assembly 11 receives the electrical stimulation signal ES and transmits it to a target region T to perform an electrical stimulation on the target region T (FIG. 2B is also referred). In other words, the electrical stimulation signal ES is transmitted to the target region T through the electrode assembly 11. In some embodiments, the electrical stimulation device 10 may be transcutaneous, transcranial, partially invasive, minimally invasive or implantable. In some embodiments, the target region T is where to be electrically stimulated by the electrical stimulation device 10, such as a central nervous system and its tissue or a peripheral nervous system and its tissue. The central nervous system and its tissue contain brain, brain stem and spinal cord; and the peripheral nervous system and its tissue contain nerves, nerve roots and root ganglia other than the brain, brain stem and spinal cord. In some embodiments, the peripheral nervous system and its tissue are functionally classified as an autonomic nervous system and a somatic nervous system, containing, for example, cranial nerves, vagus nerves, occipital nerves, trigeminal nerves, hypoglossal nerves, sphenopalatine ganglia, sacral nerves, lumbar nerves, pudendal nerves, sacral nerves, coccygeal nerves and the like.

In some embodiments, the electrode assembly 11 and the electrical stimulator 12 may be separable elements connected to each other or integrally formed. The separable electrode assembly 11 and electrical stimulator 12 are directly or indirectly coupled (e.g., clamped, snap-fit, pluggable or electrically coupled).

In some embodiments, one or more electrical stimulation devices 10 may be positioned on the target region T.

In some embodiments, the electrode assembly 11 contains an electrode of paddle-shaped, cuff-shaped, spiral, wire-shaped, thin-probe-shaped, cylindrical or the like; and the electrode assembly 11 may be linear, spiral, patch-shaped, lead-shaped, needle-shaped or the like.

FIG. 2A and FIG. 2B are a schematic diagram and a use schematic diagram of an example of the electrical stimulation device of FIG. 1. Refer to FIG. 2A and FIG. 2B. In some embodiments, the electrical stimulation device 10 may be a transcutaneous electrical stimulation device 10a. The electrode assembly 11 is embedded in one surface of the electrical stimulator 12. In use, the electrical stimulator 12 is positioned on a skin of an organism and the surface having the electrode assembly 11 is attached to the skin. In other words, both the electrical stimulator 12 and the electrode assembly 11 of the electrical stimulation device 10a are positioned outside a body of the organism. The electrical stimulation signal ES generated by the electrical stimulator 12 stimulates superficial nerves under the skin via the electrode assembly 11. In this embodiment, the target region T is the superficial nerves under the skin. The skin may be the surface of head, upper limbs, lower limbs, trunk, genitals or the like, and the superficial nerves under the skin may be nerves within about 1, 2, 3 or 4 cm subcutaneously.

In some embodiments, the electrode assembly 11 contains dual electrodes or a plurality of electrodes such that the electrical stimulation is bipolar or tripolar. In other embodiments, the electrode assembly 11 contains a working electrode and a reference electrode such that the electrical stimulation is monopolar. As shown in FIG. 2A, in a bipolar example, the electrode assembly 11 contains a pair of electrodes 111. In the electrical stimulation device 10a, the pair of electrodes 111 is positioned on the surface (i.e., a casing) of the electrical stimulator 12. In use, the electrical stimulation device 10a is attached to the skin of an arm by the electrode pair 111 and is activated to output the electrical stimulation signal ES to the electrode pair 111, so as to electrically stimulate the skin of the arm and the superficial nerves under the skin via the electrode pair 111. In some embodiments, the superficial nerves under the skin of the arm may be a musculocutaneous nerve located in biceps brachii, a radial nerve located in triceps brachii or a median nerve located near a wrist.

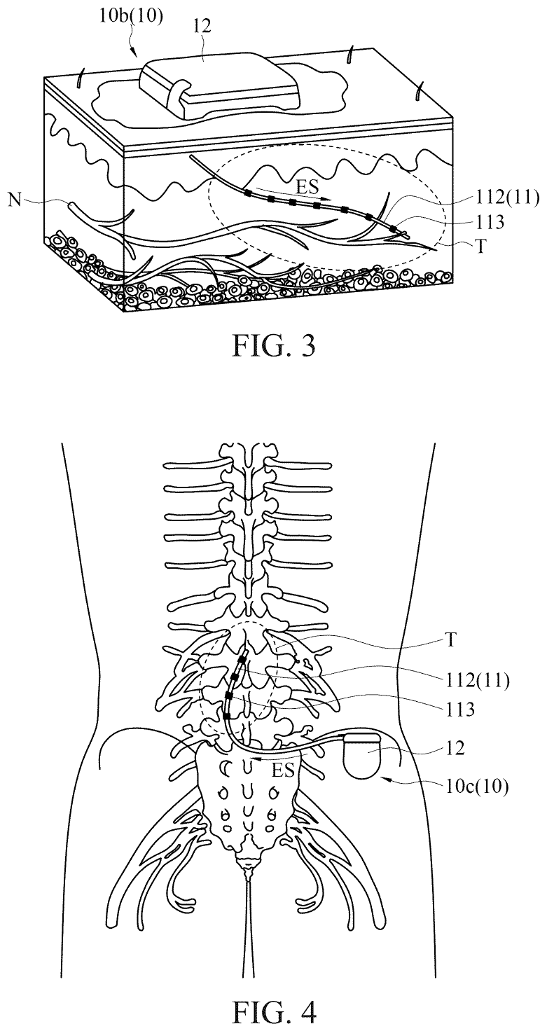

FIG. 3 is a schematic diagram of one other example of the electrical stimulation device of FIG. 1. Refer to FIG. 3. In some embodiments, the electrical stimulation device 10 may be a minimally invasive electrical stimulation device 10b. After the electrode assembly 11 is implanted, one end thereof is wirelessly or wiredly connected to the electrical stimulator 12 and the other end is implanted in the organism. In use, the electrical stimulator 12 is positioned on the skin of the organism and the electrode assembly 11 is at least partially implanted under the skin of the organism in a minimally invasive manner. In other words, the electrical stimulator 12 of the electrical stimulation device 10b is positioned outside the body of the organism, while the electrode assembly 11 of the electrical stimulation device 10b is positioned inside the body of the organism. The electrical stimulation signal ES generated by the electrical stimulator 12 wirelessly or wiredly stimulates a target nerve N near a terminal of the electrode assembly 11 (one end of the electrode assembly 11 not connected to the electrical stimulator 12) via the electrode assembly 11. In this embodiment, the target region T is the target nerve N near the terminal of the electrode assembly 11.

As shown in FIG. 3, in an example, the electrical stimulation device 10b may be a peripheral nerve electrical stimulation device, the electrode assembly 11 may be a lead 112, and the lead 112 contains a plurality of ring electrodes 113. In other embodiments, one end of the lead 112 may be implanted near the target nerve N and the other end of the lead 112 may extend over the surface of the skin to be connected with the electrical stimulator 12.

In some embodiments, the target region Tis a nerve or neural structure around the ring electrodes 113 on the lead 112. For example, the target region Tis the target nerve N around the ring electrodes 113 on the lead 112.

FIG. 4 is a schematic diagram of another example of the electrical stimulation device of FIG. 1. Refer to FIG. 4. In some embodiments, the electrical stimulation device 10 may be an implantable electrical stimulation device 10c. The electrode assembly 11 is connected to the electrical stimulator 12. In use, both the electrical stimulator 12 and the electrode assembly 11 are implanted in the organism. In other words, both the electrical stimulator 12 and the electrode assembly 11 of the electrical stimulation device 10c are positioned in vivo. In this embodiment, the target region T is a nerve near the terminal of the electrode assembly 11. As shown in FIG. 4, in an example, the electrical stimulation device 10c may be a spinal cord electrical stimulation device or a sacral nerve electrical stimulation device. In the electrical stimulation device 10c, the lead 112 (containing a plurality of ring electrodes 113) is directly connected to the electrical stimulator 12. In use, the lead 112 is implanted in the epidural space of the spine or near the sacral nerve around the buttocks of the organism.

In some embodiments, the electrical stimulation signal ES may be a biphasic electrical stimulation signal ES. In other embodiments, the electrical stimulation signal ES may also be a monophasic electrical stimulation signal ES.

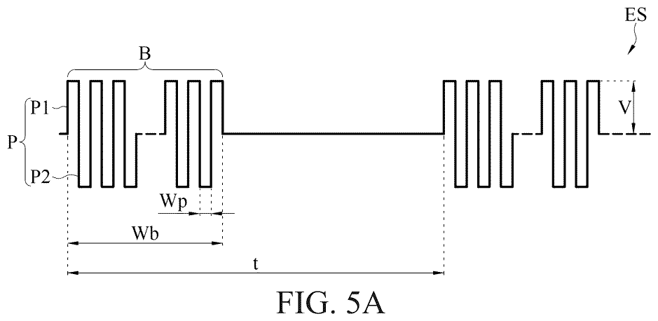

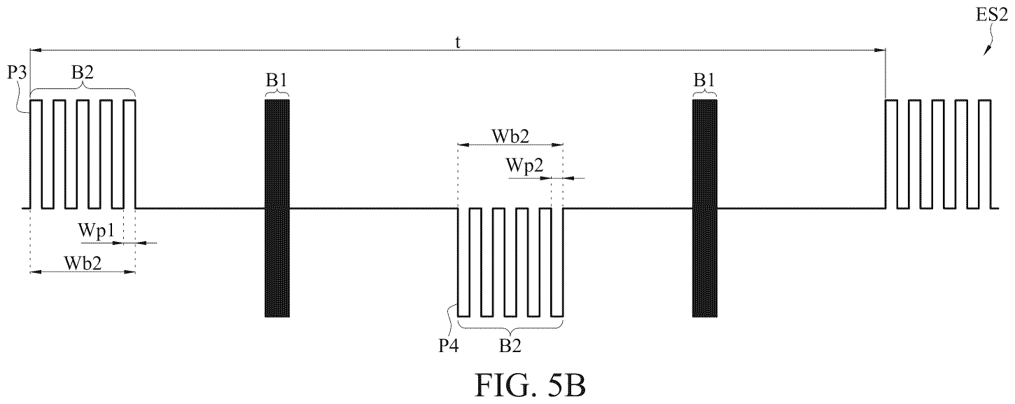

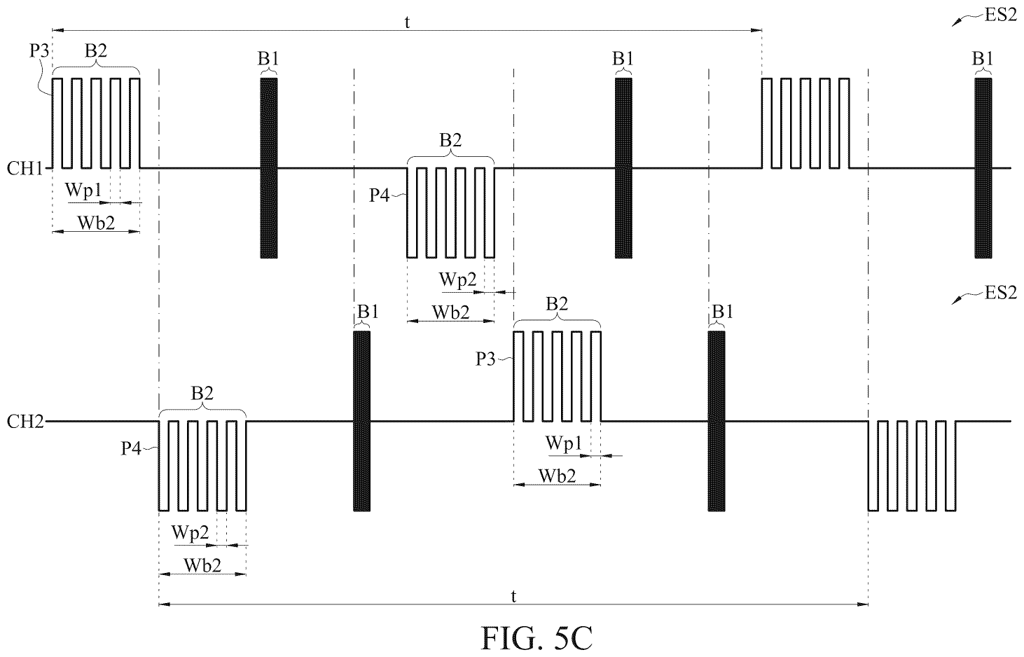

FIG. 5A is a schematic diagram of an example of the electrical stimulation signal generated by the electrical stimulator of FIG. 1. Refer to FIG. 5A. In some embodiments, the waveform of the electrical stimulation signal ES generated by the electrical stimulation device 10 is for example, biphasic, square-wave, symmetric and charge-balanced. Of course, the waveform of the electrical stimulation signal ES may be a monophasic signal, a triangular wave, a sine wave, an asymmetric signal, or a charge-unbalanced signal or the like.

In some embodiments, the electrical stimulation signal ES contains a plurality of burst signals B, and the burst signals B have the burst frequency between 0.1 Hz and 1,000 Hz. Each burst signal B contains a plurality of pulses P, and the pulses P have the pulse frequency between 20 kHz and 1,000 kHz. Preferably, the burst signals B have the burst frequency between 0.1 Hz and 500 Hz, for example, 0.1 Hz-400 Hz, 0.1 Hz-200 Hz, 0.1 Hz-100 Hz or 0.1 Hz-20 Hz. Preferably, the pulses P have the pulse frequency between 100 kHz and 750 kHz, for example, 400 kHz-600 kHz. The wave width Wb of the burst signals B is between 1 ms and 10 s, and the pulse width Wp of the pulses P is between 0.5 us and 1 ms. Preferably, the wave width Wb of the burst signals B is between 2 ms and 500 ms, for example, 20 ms. Preferably, the pulse width Wp of the pulses P is between 1 μs and 1 ms, for example, between 1 μs and 100 μs.

In some embodiments, the current intensity of the electrical stimulation signal ES may be between 0.1 mA and 120 mA, and the voltage intensity V of the electrical stimulation signal ES may be between 0.05 V and 60 V. Preferably, the current intensity is between 6 mA and 40 mA. Preferably, the voltage intensity Vis between 3 V and 20 V.

In some embodiments, the pulses P are biphasic and successive of opposite polarity. The pulses P contained in the biphasic pulses include positive pulses P1 and negative pulses P2, the positive pulses P1 and the negative pulses P2 alternately appear, and preferably, the positive pulses P1 and the negative pulses P2 are charge-balanced.

In some embodiments, the pulses P may be a square wave, a sine wave, a triangular wave or a combination thereof. In some embodiments, the burst signals B may be a square wave, a sine wave, a triangle wave or a combination thereof.

FIG. 5B is a schematic diagram of one other example of the electrical stimulation signal generated by the electrical stimulator of FIG. 1. Refer to FIG. 5B. In some embodiments, the electrical stimulation signal ES generated by the electrical stimulation device 10 is an electrical stimulation signal ES2, the electrical stimulation signal ES2 is exemplified by two alternate burst signals B1 and B2, wherein the burst signals B1 are the same as the burst signals B in FIG. 5A, and the waveform thereof is illustrated by a solid block, which is not described herein again. The burst frequency of the burst signals B2 is also between 0.1 Hz and 1,000 Hz, and preferably, the burst frequency of the burst signals B2 is less than or equal to that of the burst signals B1. Here, the burst signals B1 and B2 alternately appear at 1:1.

In some embodiments, the burst signals B2 contain a plurality of positive pulses P3 or a plurality of negative pulses P4. For example, referring to FIG. 5B, the electrical stimulation signal ES2 contains two burst signals B2, first burst signals B2 contain a plurality of positive pulses P3 and second burst signals B2 contain a plurality of negative pulses P4, the two burst signals have the same waveform but opposite phases. The pulse frequency of the positive pulses P3 and the negative pulses P4 is in a range of 20 kHz to 1,000 kHz, and preferably the pulse frequency of the positive pulses P3 and the negative pulses P4 is in the range of 20 kHz to 300 kHz. Preferably, the pulse frequency of the positive pulse P3 or the negative pulse P4 contained in the burst signals B2 is smaller than that of the pulses P contained in the burst signals B1. For example, the pulse frequency of the positive pulse P3 or the negative pulse P4 contained in the burst signals B2 is smaller than ⅓ of that of the pulses P contained in the burst signals B1.

In some embodiments, the wave width Wb2 of the burst signals B2 is also between 1 ms and 10 s, and preferably, the wave width Wb2 of the burst signals B2 is between 2 ms and 500 ms, for example, between 5 ms and 100 ms.

In some embodiments, the pulse widths Wp1 and Wp2 of the positive pulses P3 and the negative pulses P4 are also between 1 μs and 1 ms, and preferably, the pulse widths Wp1 and Wp2 of the positive pulses P3 and the negative pulses P4 are between 3.3 μs and 1 ms, for example, 1 μs. The pulse frequencies and the pulse widths Wp1 and Wp2 of the positive pulses P3 and the negative pulses P4 may be the same or different. Taking the same pulse frequencies and pulse widths as an example herein, the charge of the positive pulses P3 is equal to that of the negative pulses P4 in a unit time, thereby achieving a delayed charge balance and reducing erosion of an electrode due to polarization.

In some embodiments, when the accumulated charge of the positive pulses P3 or the negative pulses P4 of the burst signals B2 is greater than or equal to 20 μC, it can be sensible by the organism (a user using the electrical stimulation device 10), such as a jumping sense, a shaking sense or a stinging sense, such that the organism can know that the electrical stimulation is in progress.

FIG. 5C is a schematic diagram of another example of the electrical stimulation signal generated by the electrical stimulator of FIG. 1. Refer to FIG. 5C. In some embodiments, the electrical stimulation device 10 has two output ends CH1 and CH2. The two output ends CH1 and CH2 respectively output the electrical stimulation signals ES2. In this state, on periods of each burst signal B1 and B2 or each pulse P in the period time t of the electrical stimulation signal ES2 outputted by the two output ends CH1 and CH2 does not overlap with output times of the two output ends CH1 and CH2. For example, the on periods of the burst signals B1 and burst signals B2 at the output end CH1 do not overlap with those of the burst signals B1 and burst signals B2 at the output end CH2. In this embodiment, the voltage or current interference of the two output ends CH1 and CH2 when outputting the electrical stimulation signal ES2 can be reduced, thereby avoiding the instability of the outputs of the two output ends CH1 and CH2. For example, referring to FIG. 4, the electrical stimulation device 10c may contain two electrode leads 112 (FIG. 4 only shows one electrode lead 112 and the other electrode lead 112 is not shown), which respectively correspond to the two output ends CH1 and CH2 to output the electrical stimulation signal ES2 to the plurality of ring electrodes 113 on the two electrode leads 112 for bipolar output of the electrical stimulation signal ES2.

In some embodiments, the electrical stimulation signal ES generated by the electrical stimulation device 10 has the voltage intensity V and the maximum amplitude of the electrical stimulation signal ES may be ±60 V. In some embodiments, the voltage intensity Vis 3 V to 20 V. Preferably, the voltage intensity Vis 3 V to 10 V. The electrical stimulation device 10 is activated to generate the electrical stimulation signal ES and the cumulative electrical stimulation time per day is less than 12 hours.

In some embodiments, the electrical stimulation signal ES generated by the electrical stimulation device 10 does not induce activation of an upstream neural structure or a downstream neural structure of the target region T. In some embodiments, the upstream neural structure of the target region T is a cranial nerve or spinal cord. In some embodiments, the downstream neural structure of the target region T is a sciatic nerve, a sacral nerve or a peripheral nerve.

In some embodiments, the electrical stimulation signal ES generated by the electrical stimulation device 10 is transmitted to the target region T via the electrode assembly 11. But the electrical stimulation signal ES does not induce the activation of the upstream neural structure or the downstream neural structure of the target region T. For example, when the electrical stimulation device 10 is used for electrical stimulation, a target region T can be firstly selected. The target region T may be a symptomatic (e.g., pain) region, a position near the symptomatic region or on a nerve conduction pathway causing neural sensitivity or symptoms. The upstream neural structure of the target region T or the downstream neural structure of the target region T can be found by an ascending pathway and a descending pathway of the neural conduction pathway of the target region T. For example, if the target region T of the electrical stimulation signal ES is in a thoracic vertebra region of the spinal cord, the upstream neural structure is the brain, the cranial nerve or the cervical spinal region of the spinal cord, and the downstream neural structure is the sciatic nerve, the sacral nerve or the peripheral nerve of the lower limbs. If the target region T of the electrical stimulation signal ES is in the femoral nerve of the thigh, its upstream neural structure is the brain, cranial nerve, spinal cord, sciatic nerve or sacral nerve, and its downstream neural structure is a peripheral nerve of a calf or a foot plate.

The upstream neural structure is outside 3 cm of radius of the target region T and the downstream neural structure is outside 3 cm of radius of the target region T.

In some embodiments, the activation of the upstream neural structure is generating an action potential from the upstream neural structure and the activation of the downstream neural structure is generating an action potential from the downstream neural structure. In some embodiments, the electrical stimulation signal ES generated by the electrical stimulation device 10 does not induce the action potential to transmit a neural signal to the upstream or downstream neural structure. The upstream neural structure and/or the downstream neural structure include a sensory nerve and a motor nerve.

In some embodiments, the electrical stimulation signal ES generated by the electrical stimulation device 10 also do not activate a muscle fiber of the target region T.

In some embodiments, the neurons may be classified into the sensory nerve and motor nerve according to their functions. The electrical stimulation signal ES generated by the electrical stimulation device 10 does not activate the sensory nerve and/or motor nerve of the upstream and downstream neural structures, nor induce the sensory nerve and/or motor nerve of the upstream and downstream neural structures to generate the action potential. In other words, any neuron is not activated by the electrical stimulation signal ES generated by the electrical stimulation device 10 to generate the action potential.

In some embodiments, a nerve fiber can be classified into A fibers (including Aα and Aβ), B fibers and C fibers according to its thickness and conduction velocity. The electrical stimulation signal ES generated by the electrical stimulation device 10 does not activate the A fibers, B fibers and C fibers of the upstream and downstream neural structures, nor induce the A fibers, B fibers and C fibers of the upstream and downstream neural structures to generate the action potential. In other words, any nerve fiber is not activated by the electrical stimulation signal ES generated by the electrical stimulation device 10 to generate the action potential.

In some embodiments, the electrical stimulation signal ES generated by the electrical stimulation device 10 desensitizes nerves. In some embodiments, the electrical stimulation signal ES generated by the electrical stimulation device 10 has the voltage threshold (minimum voltage with therapeutic effect) sufficient to desensitizes nerves. In some embodiments, under the voltage threshold, the electrical stimulation signal ES generated by the electrical stimulation device 10 does not induce the activation of the upstream or downstream neural structure of the target region T and can desensitizes nerves. In some embodiments, the electrical stimulation signal ES generated by the electrical stimulation device 10 has effect of desensitizing nerves, thereby inhibiting and/or relieving acute pain, chronic pain, overactive bladder (OAB), premature ejaculation, or diseases or symptoms caused by nerve sensitization.

In some embodiments, when the electrical stimulation signal ES generated by the electrical stimulation device 10 has the voltage intensity V greater than 1.5 times of the voltage threshold, the electrical stimulation signal ES still does not induce the activation of the upstream or downstream neural structure of the target region T. In some embodiments, when the electrical stimulation signal ES generated by the electrical stimulation device 10 has the voltage intensity V greater than 1.5 times of the voltage threshold, the electrical stimulation signal ES still does not induce the activation of the upstream or downstream neural structure of the target region T and can inhibit and/or relieve pain, relieve symptoms and/or desensitizes nerves.

In some embodiments, the electrical stimulation signal ES generated by the electrical stimulation device 10 inhibits and/or relieves pain. In some embodiments, the electrical stimulation signal ES inhibits and/or relieves local pain. In some embodiments, the electrical stimulation signal ES inhibits and/or relieves chronic pain.

In some embodiments, the electrical stimulation signal ES generated by the electrical stimulation device 10 inhibits nerve pain, for example, neuropathic pain or nerve pain caused by nerve injury.

In some embodiments, the electrical stimulation signal ES generated by the electrical stimulation device 10 inhibits mechanical hypersensitivity induced by the nerve injury and reduces the activity of brain neurons.

In some embodiments, the electrical stimulation signal ES generated by the electrical stimulation device 10 is a paresthesia-free electrical stimulation signal or a subthreshold electrical stimulation signal, wherein the subthreshold electrical stimulation signal is an electrical stimulation signal that fails to enable the organism (the user using the electrical stimulation device 10) to generate the action potential.

### Example I: Animal Test-Mechanical Hypersensitivity Test

### A. Test Process:

### (1) Brain and Spinal Cord Electrode Implantation Procedure

Electrodes were implanted in the brain and spinal cord of normal (Sprague Dawley, SD) rats. The electrodes implanted in the brain were recording electrodes and the electrodes implanted in the spinal cord were electrical stimulation electrodes. Besides, the rats treated by the electrode implantation procedure were divided into an experimental group A, an experimental group B and an experimental group C with 4-5 rats in each group.

Implanting the electrodes in the brain referred to implanting the electrodes in bilateral primary somatosensory cortex of the hind limb (S1HL) and anterior cingulate cortex (ACC). Implanting the electrodes in the spinal cord referred to implanting bipolar spinal cord electrical stimulation electrodes (GIMER Medical, Taiwan) in dorsal epidural space of the spinal cord at the thoracic vertebrae (T9-T11) of the rats. Leads with electrodes connected to the bipolar spinal cord electrical stimulation electrodes were connected to an electrical stimulator (ES1001, GIMER Medical, Taiwan) in vitro. When the electrical stimulator generated an electrical stimulation signal, the electrical stimulation signal was transmitted to the bipolar spinal cord electrical stimulation electrodes through the leads.

The somatosensory cortex and anterior cingulate cortex played important roles in central nociception. The somatosensory cortex was involved in coding sensory characteristics of pain, such as the nature (tingling, burning or pain), location and duration of the pain, while the anterior cingulate cortex controlled emotional and motivational responses, and was involved in the emotional and situational aspects of the pain.

### (2) Nerve Injury Treatment

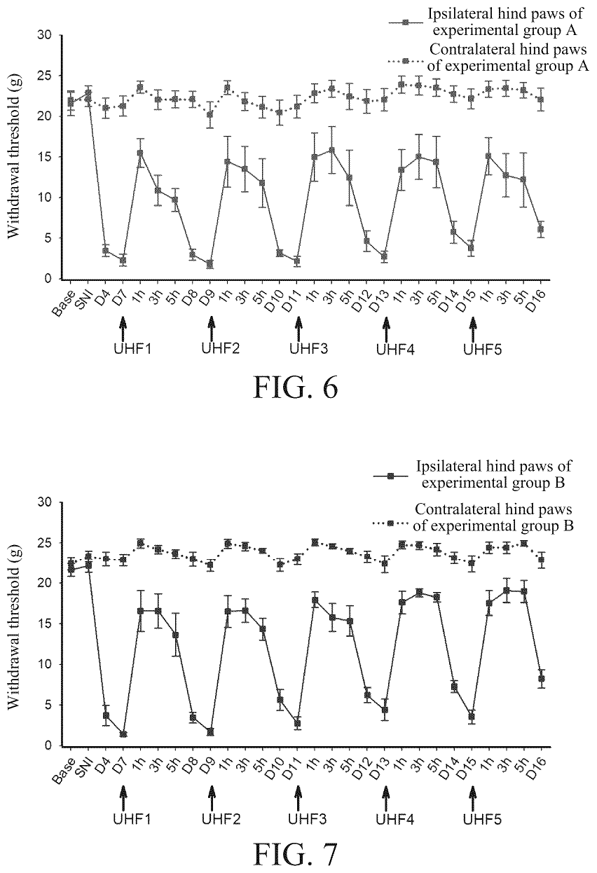

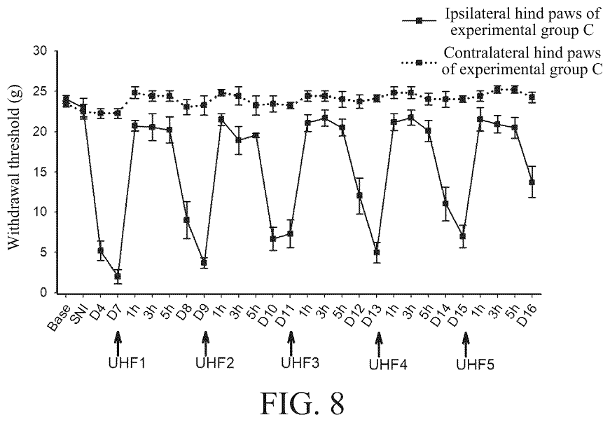

After 7 to 10 days of the brain and spinal cord electrode implantation treatment, tibial nerves and common peroneal nerves of left hind paws of the rats in each group were ligated with 4-0 silk and cut at distance of about 2 mm from the ligation sites as nerve injury. In FIGS. 6-8, represented by spared nerve injury (SNI).

(3) Mechanical Hypersensitivity Test after Nerve Injury Treatment

After the nerve injury treatment of the rats in each group, a monofilament tactile pain measurement (Von Frey) was performed on the rats in each group using a Von Frey filament (Stoelting, Wood Dale, IL) to evaluate mechanical hypersensitivity and calculate withdrawal thresholds of the rats in each group. Baselines (represented as Base in FIGS. 6-8) of the withdrawal thresholds of each group were the withdrawal thresholds measured at least 2 days prior to the nerve injury treatment.

### (4) Ultrahigh Frequency Electrical Stimulation Treatment

After 7, 9, 11, 13 and 15 days of the nerve injury treatment of the rats in each group (represented by D7, D9, D11, D13 and D15 in FIGS. 6-8), the rats in each group were treated with different durations of ultrahigh frequency electrical stimulation signals. The ultrahigh frequency electrical stimulation signal contained at least one burst signal, and the burst signal contained pulses. The pulses were biphasic sine waves. The wave width of the burst signal was 25 ms and the burst frequency of the burst signal was 2 Hz. The pulse width of the pulses was 2 μs, the pulse frequency of the pulses was 500 kHz, and the voltage of the electrical stimulation signal was ±3 V. The rats in the experimental groups A, B and C were all subjected to the ultrahigh frequency electrical stimulation treatment for 6 times (once every two days). The difference was that the duration of the ultrahigh frequency electrical stimulation treatment of the rats in the experimental group A was 5 minutes each time; the duration of the ultrahigh frequency electrical stimulation treatment of the rats in the experimental group B was 10 minutes each time; and the duration of the ultrahigh frequency electrical stimulation treatment of the rats in the experimental group C was 20 minutes each time. In FIGS. 6-8, UHF1 to UHF6 represented the 1st to 6th ultrahigh frequency electrical stimulation treatments respectively.

(5) Mechanical Hypersensitivity Test after Ultrahigh Frequency Electrical Stimulation Treatment

After the ultrahigh frequency electrical stimulation treatment of the rats in each group, a monofilament tactile pain measurement (Von Frey) was performed on the rats in each group using a Von Frey filament (Stoelting, Wood Dale, IL) to evaluate the mechanical hypersensitivity and calculate the withdrawal thresholds of the rats in each group. The analgesic effect was evaluated by Von Frey filament-induced allodynia. The mechanical hypersensitivity was a nerve-sensitive reaction.

### (6) Statistical Analysis

The withdrawal thresholds of the rats in each group were analyzed by a two-way repeated measures ANOVA and a post hoc analysis was performed by a multiple comparison method (Tukey method). Experimental data were presented as mean±standard deviation (SD). All the analyses were performed using a two-tailed test. P<0.05 was statistically significant.

### B. Test Results:

Refer to FIGS. 6-8. After the rats in each group were subjected to the nerve injury treatment (SNI), the withdrawal thresholds of the left hind paws (nerve injury ipsilateral hind paws) of the rats in each group sharply decreased from the baseline (Base) 21.9±1.7 g to 1.0±0.3 g and remained for at least one month. The mechanical hypersensitivity generated after the nerve injury treatment was a chronic neuropathic pain.

Refer to FIGS. 6-8 continuously. 5 hours after the rats in the experimental groups A, B and C were subjected to the ultrahigh frequency electrical stimulation treatment, the withdrawal thresholds were respectively significantly increased to 13.3±6.5 g, 18.5±3.6 g and 21.0±2.8 g. On the 2nd day after the ultrahigh frequency electrical stimulation treatment, the withdrawal thresholds of the rats in the experimental groups A, B and C were increased to 6.1±2.5 g, 8.2±2.7 g and 14.3±5.9 g respectively. In other words, after the ultrahigh frequency electrical stimulation treatment (pulse frequency of 500 kHz and voltage of ±3 V), the withdrawal thresholds of the left hind paws of the rats in each group were significantly increased, indicating that the voltage 3 V was the electrical stimulation intensity with a therapeutic effect. The ultrahigh frequency electrical stimulation treatment significantly inhibited the mechanical hypersensitivity of the left hind paws induced by the nerve injury. The effect of increasing the withdrawal thresholds occurred 1 hour after the ultrahigh frequency electrical stimulation treatment, continued for at least 5 hours, and decreased on the 2nd day after the ultrahigh frequency electrical stimulation treatment.

From the above results, it can be seen that the withdrawal thresholds at 5 hours and on the 2nd day in the experimental group C with longer ultrahigh frequency electrical stimulation treatment (duration of 20 minutes each time) were higher than those in the experimental group A (duration of 5 minutes each time) and the experimental group B (duration of 10 minutes each time). Increasing the duration from 5 minutes to 20 minutes significantly enhanced the effect of increasing the withdrawal thresholds. In other words, the longer duration of the ultrahigh frequency electrical stimulation had a stronger effect on inhibiting pain and was significantly time dependent.

Besides, from the above results, it can be seen that the repeated stimulation produced an analgesic effect of similar duration and effectiveness. Stimulation repeated every 2 days for 20 minutes 1 time will gradually enhance the analgesic effect of the effectiveness. The analgesic effect was gradually enhanced after 5 times of 20 minutes of the ultrahigh frequency electrical stimulation treatment and the duration of analgesia was also gradually prolonged. For example, the withdrawal thresholds of the rats in the experimental group C after 1 time of 20-minute ultrahigh frequency electrical stimulation treatment was 8.0±4.3 g, while the withdrawal thresholds after 5 times of the 20-minute ultrahigh frequency electrical stimulation treatment was 14.3±5.9 g (not shown in the figure). In other words, the ultrahigh frequency electrical stimulation treatment had the effects of short stimulation time and long-acting analgesia. The ultrahigh frequency electrical stimulation signal generated by the electrical stimulation device inhibited the mechanical hypersensitivity induced by the nerve injury.

### Example II: Animal Test-Stimulation Threshold Test

### A. Test Process:

### (1) Brain, Spinal Cord and Sciatic Nerve Electrode Implantation Procedure

Electrodes were implanted in the brain and spinal cord of normal (Sprague Dawley, SD) rats. The electrodes implanted in the brain were recording electrodes and the electrodes implanted in the spinal cord were electrical stimulation electrodes. Then 7 days after the implantation of the electrodes, the recording electrodes were implanted in sciatic nerves region of the rats in which the electrodes had been implanted. Besides, the rats treated by the electrode implantation were divided into an experimental group D, an experimental group E, an experimental group F, an experimental group G and an experimental group H with 4-5 rats in each group.

Implanting the electrodes in the brain referred to implanting the electrodes in bilateral primary somatosensory cortex of the hind limb (S1HL) and anterior cingulate cortex (ACC). Implanting the electrodes in the sciatic nerves region referred to implanting the electrodes around the nerves in the middle third of the sciatic nerves on the posterior lateral side of the right thigh. Implanting the electrodes in the spinal cord referred to implanting bipolar spinal cord electrical stimulation electrodes (GIMER Medical, Taiwan) in the dorsal epidural space of the spinal cord at the thoracic vertebrae (T9-T11) of the rats. Leads with electrodes connected to the bipolar spinal cord electrical stimulation electrodes were connected to an electrical stimulator (ES1001, GIMER Medical, Taiwan) in vitro. After the electrical stimulator generated an electrical stimulation signal, the electrical stimulation signal was transmitted to the bipolar spinal cord electrical stimulation electrodes through the leads.

### (2) Electrical Stimulation Test and Activation Threshold Recording

After the electrodes were implanted in the rats in each group, the rats in each group were given different frequency electrical stimulation signals through a spinal cord electrical stimulation. A local field potential was measured in the brain and a compound action potential was measured in the sciatic nerves to obtain activation thresholds on the brain and sciatic nerves. As the intensity of the electrical stimulation signal gradually increased, when the electrodes measured the minimum intensity (voltage) value of the electrical stimulation signal transmitted from the spinal cord, it was the activation threshold that can cause the action potential, also called stimulation threshold. Therefore, the stimulation thresholds of different frequency electrical stimulation signals for causing cranial nerve and sciatic nerve reactions can be measured. The local field potential was the sum of electrical activities of a plurality of neurons in a local regions of the electrode terminal implanted in the brain and was mostly related to the action potential. The local field potential measured during the stimulation state (electrical stimulation) was also known as an evoked potential. The compound action potential was the sum of the action potentials of a plurality of nerves.

The parameters of the electrical stimulation signals for testing the stimulation thresholds in each group were respectively as follows:

Experimental group D (0.1 Hz, single pulse): the pulse frequency of the electrical stimulation signal was 0.1 Hz, the pulse width was 2 ms, and the electrical stimulation signal only contained a 1 ms positive phase square wave and a 1 ms negative phase square wave. The positive wave and the negative wave of the experimental groups D to G were generated directly and sequentially to achieve charge balance, which was not described in detail below.

Experimental group E (50 Hz, single pulse/pulse train): represented a conventional low frequency electrical stimulation signal, the pulse frequency of the electrical stimulation signal was 50 Hz, and the pulse width was 400 μs. The electrical stimulation signal in the form of the single pulse only contained a 200 μs positive phase square wave and a 200 μs negative phase square wave. The electrical stimulation signal in the form of the pulse train was the single pulse that is applied once every other 19.6 ms. After the electrical stimulation signal was applied, if no stimulation threshold was recorded after observation, the voltage was increased to continue the electrical stimulation, and the step was repeated until the stimulation threshold was observed.

Experimental group F (10 kHz, single pulse/pulse train): represented a high frequency electrical stimulation signal, the pulse frequency of the electrical stimulation signal was 10 kHz, and the pulse width was 40 μs. The electrical stimulation signal in the form of the single pulse only contained a 20 μs positive phase sine wave and a 20 μs negative phase sine wave. The electrical stimulation signal in the form of the pulse train was the single pulse that is applied once every other 60 μs. After the electrical stimulation signal was applied, if no stimulation threshold was recorded after observation, the voltage was increased to continue the electrical stimulation, and the step was repeated until the stimulation threshold was observed.

Experimental group G (500 kHz, single pulse/single pulse train): represented an ultrahigh frequency electrical stimulation signal, the pulse frequency of the electrical stimulation signal was 500 kHz, and the pulse width was 2 μs. The electrical stimulation signal in the form of the single pulse only contained a 1 μs positive phase sine wave and a 1 μs negative phase sine wave. The electrical stimulation signal in the form of the single pulse train was continuously applied the single pulse and continued for 25 ms. After the electrical stimulation signal was applied, if no stimulation threshold was recorded after observation, the voltage was increased to continue the electrical stimulation, and the step was repeated until the stimulation threshold was observed.

Experimental group H (500 kHz, continuous pulse train): represented a continuous ultrahigh frequency electrical stimulation signal, the electrical stimulation signal contained a plurality of burst signals at the burst frequency of 5 Hz and the wave width of 25 ms, and each burst signal contained a plurality of pulses at the pulse frequency of 500 kHz and the pulse width of 2 μs. The pulse contained a 1 μs positive phase sine wave and a 1 μs negative phase sine wave. The electrical stimulation signal in the form of the continuous pulse train was the single pulse train that is applied once every other 175 ms. After the electrical stimulation signal was applied, if no stimulation threshold was recorded after observation, the voltage was increased to continue the electrical stimulation, and the step was repeated until the stimulation threshold was observed.

The electrical stimulation was performed according to different parameters of each group to measure the minimum intensity (voltage) that excited the local field potential in the cerebral cortex and the compound action potential in the sciatic nerve to determine whether the electrical stimulation signal of each group can be transmitted orthodromic (ascending) to the brain or spinal cord or antidromic (descending) to downstream or peripheral nerves. In other words, a detectable local field potential elicited in the cerebral cortex or a detectable compound action potential elicited in the sciatic nerve of each group were measured as a stimulation threshold for eliciting neuron responses of the cranial nerve and sciatic nerve.

### B. Test Results:

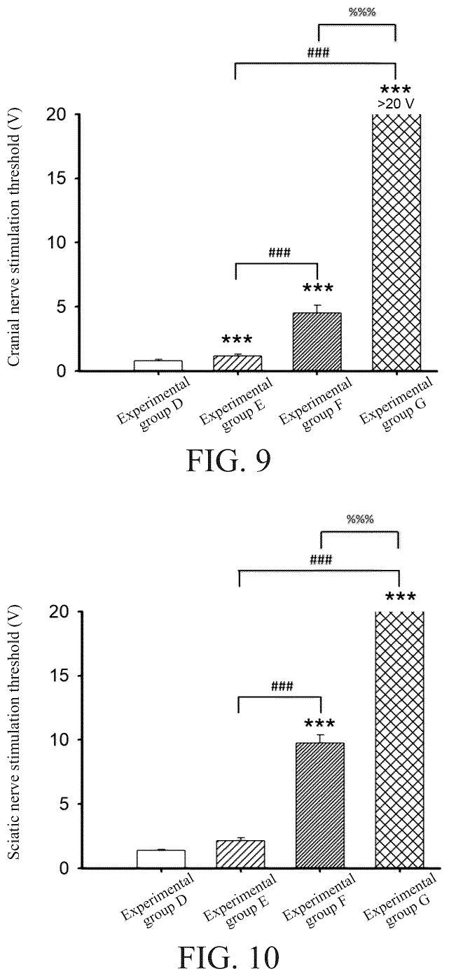

Refer to FIG. 9. When the electrical stimulation signal was applied in the form of the single pulse, the cranial nerve stimulation threshold (minimum intensity of brain activation) of the experimental group D (0.1 Hz) was about 0.8±0.2 V; the cranial nerve stimulation threshold of the experimental group E (50 Hz) was about 1.2±0.3 V; the cranial nerve stimulation threshold of the experimental group F (10 kHz) was about 4.5±1.4 V; and the cranial nerve stimulation threshold of the experimental group G (500 kHz) was >20 V. It can be seen from FIG. 9 that when the pulse frequency of the electrical stimulation was higher, the stimulation threshold of the electrical stimulation to the cranial nerves was higher.

Refer to FIG. 10. When the electrical stimulation signal was applied in the form of the single pulse, the test results of the sciatic nerve stimulation thresholds (minimum intensity of sciatic nerve activation) of the experimental groups D to G (0.1 Hz, 50 Hz, 10 kHz, and 500 kHz, respectively) were similar to the trend of the cranial nerve stimulation thresholds. Each sciatic nerve stimulation threshold was higher than the cranial nerve stimulation threshold.

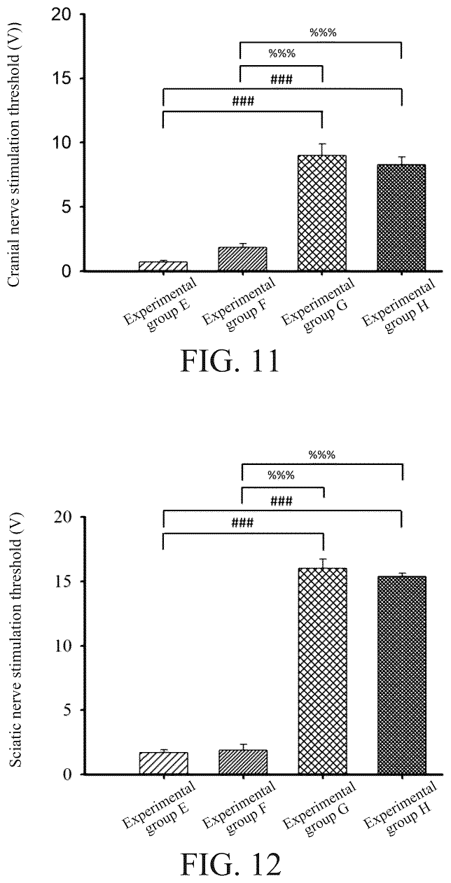

FIG. 11 (cranial nerve) and FIG. 12 (sciatic nerve) were referred and respectively represented the test results of the pulse trains of the experimental group E (50 Hz) and the experimental group F (10 kHz), and the test results of the experimental group G (500 kHz, single pulse train) and the experimental group H (500 kHz, continuous pulse train). As shown in FIG. 11, when the 500 kHz ultrahigh frequency electrical stimulation was applied, the cranial nerve stimulation threshold was approximately the same in the test results of either a single pulse train or a continuous pulse train. But compared with other test results of lower frequencies of 50 Hz and 10 kHz, the test results had a significant difference. The cranial nerve stimulation threshold of the 500 kHz ultrahigh frequency electrical stimulation was significantly greater than the cranial nerve stimulation threshold of the lower frequencies of 50 Hz and 10 kHz. As shown in FIG. 12, when the 500 kHz ultrahigh frequency electrical stimulation was applied, the sciatic nerve stimulation threshold was approximately the same in the test results of either a single pulse train or a continuous pulse train. But compared with other test results of lower frequencies of 50 Hz and 10 kHz, the test results had a significant difference. The sciatic nerve stimulation threshold of the 500 kHz ultrahigh frequency electrical stimulation was significantly greater than the sciatic nerve stimulation threshold of the lower frequencies of 50 Hz and 10 kHz.

The minimum voltage intensity for activating the cranial or sciatic nerve of the ultrahigh frequency electrical stimulation signal (20 kHz-1,000 kHz) was both much greater than that of the low frequency electrical stimulation signal (1 Hz-500 Hz) and the high frequency electrical stimulation signal (1 kHz-10 kHz). Thus, it was confirmed that the ultrahigh frequency electrical stimulation signal was a localized electrical stimulation signal, did not activate the upstream or downstream neural structure, but still achieved the effects of relieving pain, relieving symptoms, or desensitizing overactive nerves, and thus provided a great guarantee for the safety of an organism receiving the electrical stimulation.

In addition to this, when the electrical stimulation signal generated by the electrical stimulation device had the voltage intensity greater than 1.5 times of the voltage threshold (minimum voltage with therapeutic effect), the electrical stimulation signal still did not induce the activation of the upstream or downstream neural structure of the target region and can inhibit and/or relieve pain, relieve symptoms and/or desensitizes nerves. When the electrical stimulation signal generated by the electrical stimulation device had the voltage intensity greater than 1.5 times to 5 times (1.5 times, 2 times, 2.5 times, 3 times, 3.5 times, 4 times, 4.5 times or 5 times) the voltage threshold, the electrical stimulation signal still did not induce the activation of the upstream or downstream neural structure of the target region and can inhibit and/or relieve pain, relieve symptoms and/or desensitizes nerves. For example, when the voltage threshold was 3 V, i.e. the minimum voltage with a therapeutic effect was 3 V (the voltage of the voltage threshold may be different according to the application situation), when the ultrahigh frequency electrical stimulation was performed and the voltage intensity of the electrical stimulation signal was 1.5 times of the voltage threshold (4.5 V), the activation of the upstream or downstream neural structure of the target region still cannot be induced, but the activation of the neural structure within 3 cm radius of the target region was induced.

In addition, when the ultrahigh frequency electrical stimulation was performed under the voltage intensity (the voltage larger than the voltage threshold) with a therapeutic effect, the daily cumulative time of the electrical stimulation signal generated by using the electrical stimulation device was less than 12 hours per day. For example, the electrical stimulation device was used 5 minutes to 60 minutes each time and 1 to 12 times per day. From the above results, it can be seen that the ultrahigh frequency electrical stimulation generated by the electrical stimulation device did not induce orthodromic or antidromic neural signal transduction. Under the condition that the ultrahigh frequency electrical stimulation generated by the electrical stimulation device did not cause orthodromic or antidromic nerve signal conduction, the ultrahigh frequency electrical stimulation generated by the electrical stimulation device did not influence a spinal cord loop and did not cause spinal cord injury or nerve function defect.

### Example III: Clinical Test

A clinical test was a double-blind and double-arm trial. Enrolled subjects with carpal tunnel syndrome were randomly divided into a test group (28 subjects) and a placebo group (30 subjects). The subjects in both the test and placebo groups were fitted with a StimOn electrical stimulation device (GIMER Medical, Taiwan) on the median nerves of the wrists to receive a transcutaneous electrical stimulation at the wrists. The median nerves of the wrists were a peripheral nerve.

The subjects in the test group received an electrical stimulation signal generated by the electrical stimulation device, the electrical stimulation signal contained a plurality of burst signals, the burst frequency was 2 Hz, each burst signal contained a plurality of pulses, the pulse frequency was 500 kHz, and the voltage intensity of the electrical stimulation signal was about ±3.0 V. The duration of the electrical stimulation signal was 15 minutes. The subjects in the placebo group did not receive an electrical stimulation signal. During the electrical stimulation, the subjects were asked whether or not there were any paresthesia including shaking, pinching, or tapping. In other words, the subjects were asked during the electrical stimulation whether or not the subjects felt that the electrical stimulation was being performed.

Refer to table 1. There was no significant difference between the two groups. In other words, the ultrahigh frequency electrical stimulation signal generated by the electrical stimulation device only acted on a local region, only generated a regional local potential and did not induce an action potential to be transmitted to the brain to generate perception. Therefore, the subjects cannot perceive the generation of the electrical stimulation signal.

TABLE 1

| | | Test group | | Placebo group | | Sum | |
|---|---|---|---|---|---|---|---|
| Parameters | | (N = 28) | | (N = 30) | | (N = 58) | |
| Stimulation finished | | 28 | (100%) | 30 | (100%) | 58 | (100%) |
| Paresthesia | None | 24 | (85.7%) | 24 | (80.0%) | 48 | (82.8%) |
| during | Yes, | 4 | (14.3%) | 5 | (16.7%) | 9 | (15.5%) |
| therapy | endurable | | | | | | |
| | Yes, painful | 0 | | 1 | (3.3%) | 1 | (1.7%) |
| Paresthesia | Mean value | 0.9 | (2.35) | 0.8 | (1.91) | 0.8 | (2.12) |
| scores | (standard | | | | | | |
| | deviation) | | | | | | |
| Subjects | Not know | 24 | (85.7%) | 24 | (80.0%) | 48 | (82.8%) |
| considered | Placebo | 0 | | 2 | (6.7%) | 2 | (3.4%) |
| to be | group | | | | | | |
| assigned | Test group | 4 | (14.3%) | 4 | (13.3%) | 8 | (13.8%) |

| | | |
|---|---|---|
| into groups | p value | 0.1250 |

A conventional low frequency electrical stimulation exceeds a neural stimulation threshold to induce an action potential and transmit a signal to the brain, such that the organism perceives the electrical stimulation signal and masks the feeling of pain. On the contrary, the ultrahigh frequency electrical stimulation generated by the electrical stimulation device does not induce an action potential to be transmitted to the brain so as to generate perception. In other words, the ultrahigh frequency electrical stimulation generated by the electrical stimulation device is a “paresthesia-free” electrical stimulation, a “paresthesia-independent” electrical stimulation, a “subthreshold stimulation” electrical stimulation or a “sub-perception” electrical stimulation.

In summary, the electrical stimulation device or method does not induce the activation of the upstream or downstream neural structure of the target region. Under the voltage intensity with a therapeutic effect, the electrical stimulation device or method does not cause the activation of an orthodromic or antidromic neural structure, does not influence a spinal cord loop, does not cause spinal cord injury, does not cause myoelectric signal conduction, and does not cause neurological function defect. The electrical stimulation signal of the electrical stimulation device or method is a “paresthesia-free” electrical stimulation signal, a “paresthesia-independent” electrical stimulation signal, a “subthreshold stimulation” electrical stimulation signal or a “sub-perception” electrical stimulation signal.

What is claimed is:

1. An electrical stimulation device, comprising
an electrode assembly; and
an electrical stimulator generating an electrical stimulation signal and coupled to the electrode assembly,
wherein the electrical stimulation signal is transmitted to a target region via the electrode assembly and contains a plurality of burst signals, the burst signals have the burst frequency between 0.1 Hz and 1,000 Hz, each the burst signal contains a plurality of pulses, the pulses have the pulse frequency between 20 kHz and 1,000 kHz, and the electrical stimulation signal does not induce activation of an upstream neural structure or a downstream neural structure of the target region.

2. The electrical stimulation device according to claim 1, wherein the activation of the upstream neural structure is generating an action potential from the upstream neural structure and the activation of the downstream neural structure is generating an action potential from the downstream neural structure.

3. The electrical stimulation device according to claim 1, wherein the upstream neural structure of the target region is a cranial nerve or spinal cord.

4. The electrical stimulation device according to claim 1, wherein the downstream neural structure of the target region is a sciatic nerve, a sacral nerve or a peripheral nerve.

5. The electrical stimulation device according to claim 1, wherein the electrical stimulation signal has a voltage threshold sufficient to desensitizes nerves.

6. The electrical stimulation device according to claim 5, wherein when the electrical stimulation signal has a voltage intensity greater than 1.5 times of the voltage threshold, the electrical stimulation signal still does not induce the activation of the upstream neural structure or the downstream neural structure of the target region.

7. The electrical stimulation device according to claim 6, wherein the electrical stimulation signal has the pulse frequency between 100 kHz and 750 kHz.

8. The electrical stimulation device according to claim 1, wherein the upstream neural structure or the downstream neural structure is outside 3 cm radius of the target region.

9. The electrical stimulation device according to claim 1, wherein a cumulative time of the electrical stimulation signal is less than 12 hours per day.

10. An electrical stimulation method, comprising:
coupling an electrode assembly to a target region;
generating an electrical stimulation signal from an electrical stimulator, wherein the electrical stimulator couples to the electrode assembly, the electrical stimulation signal contains a plurality of burst signals, the burst signals have the burst frequency between 0.1 Hz and 1,000 kHz, each the burst signal contains a plurality of pulses, and the pulses have the pulse frequency between 20 kHz and 1,000 kHz; and
transmitting the electrical stimulation signal to the electrode assembly to electrically stimulate the target region, wherein the electrical stimulation signal does not induce activation of an upstream neural structure or a downstream neural structure of the target region.

11. The method according to claim 10, wherein the activation of the upstream neural structure is generating an action potential from the upstream neural structure and the activation of the downstream neural structure is generating an action potential from the downstream neural structure.

12. The method according to claim 10, wherein the upstream neural structure of the target region is a cranial nerve or spinal cord.

13. The method according to claim 10, wherein the downstream neural structure of the target region is a sciatic nerve, a sacral nerve or a peripheral nerve.

14. The method according to claim 10, wherein the electrical stimulation signal has a voltage threshold sufficient to desensitizes nerves.

15. The method according to claim 14, wherein when the electrical stimulation signal has a voltage intensity greater than 1.5 times of the voltage threshold, the electrical stimulation signal still does not induce the activation of the upstream neural structure or the downstream neural structure of the target region.

16. The method according to claim 15, wherein the electrical stimulation signal has the pulse frequency between 100 kHz and 750 kHz.

17. The method according to claim 10, wherein the upstream neural structure or the downstream neural structure is outside 3 cm radius of the target region.

18. The method according to claim 10, wherein the electrical stimulation signal inhibits and/or relieves pain.

19. The method according to claim 10, wherein a cumulative time of the electrical stimulation signal is less than 12 hours per day.
